(19) United States
(12) Patent Application Publication
(10) Pub. No.: US 2021/0346416 A1
(43) Pub. Date: Nov. 11, 2021

(54) Pharmaceutical Compositions and Methods for their Use in Antiviral Therapy

(71) Applicant: Ralph Lipp, Palmetto Bay, FL (US)

(72) Inventor: Ralph Lipp, Palmetto Bay, FL (US)

(21) Appl. No.: 17/245,841

(22) Filed: Apr. 30, 2021

Related U.S. Application Data
(60) Provisional application No. 62/704,341, filed on May 5, 2020.
(60) Provisional application No. 63/089,283, filed on Oct. 8, 2020.

(51) Int. Cl. A61K 31/7048; A61K 9/00; A61K 31/245; A61K 31/675; A61K 31/4045; A61K 31/137

(52) U.S. Cl. CPC A61K 31/7048; A61K 9/0073; A61K 9/0043; A61K 31/137; A61K 31/675; A61K 31/4045; A61K 31/245

(57) ABSTRACT

Pharmaceutical compositions comprise at least one active agent and a pharmaceutically acceptable carrier for application to the respiratory tract. The active agents are from the area of antivirals. The pharmaceutical compositions can be used for application to the respiratory tract in order to treat virus infections including COVID-19.

### RELATED U.S. APPLICATION DATA

Provisional application No. 62/704,341, filed on May 5, 2020

Provisional application No. 63/089,283, filed on Oct. 8, 2020

### BACKGROUND OF THE INVENTION

### 1. Field of the Invention

The present invention relates to pharmaceutical compositions and methods for their use in antiviral therapy. The compositions comprise one or more active agents.

### 2. Description of the Background Art

The virus SARS-CoV-2 (severe acute respiratory syndrome coronavirus 2; of the genus Betacoronavirus) is the causative agent of COVID-19 (a mild to severe respiratory illness). COVID-19 is mainly transmitted by contact with infectious material, including respiratory droplets. It is characterized by fever, cough, and shortness of breath and can progress to pneumonia and respiratory failure. COVID-19 was first identified in 2019. By April 22, 2020 the COVID-19 Dashboard by the Center for Systems Science and Engineering (CSSE) at Johns Hopkins University (JHU) reported over 2.6 million confirmed cases of COVID-19 and over 181,000 deaths on a global basis, thereby affecting 185 countries/regions (https://coronavirus.jhu.edu/map.html; accessed Apr. 22, 2020).

As such, there is currently a significant unfulfilled need for therapies to prevent and/or treat COVID-19.

Vaccination has been used as the main intervention to protect humans against virus infections for many years. In addition to vaccines, several FDA-approved pharmaceutical compositions comprising antiviral agents are being used to treat certain virus infections today. However, there is still a significant unfulfilled need for pharmaceutical compositions which can be used to effectively prevent and/or treat infections with SARS coronaviruses.

SARS (severe acute respiratory syndrome) is caused by the SARS coronavirus (SARS-CoV) which first infected humans in 2002 and was identified in 2003. The SARS epidemic affected 26 countries and resulted in more than 8,000 cases since 2003 (https://www.who.int/ith/diseases/sars/en/; accessed Apr. 23, 2020). SARS-CoV belongs to the same virus family as SARS CoV-2 and, despite significant research efforts since 2003, no approved antiviral treatment for the therapy of SARS is currently available. This shows that the development of treatments against infections with SARS coronaviruses is challenging and that failures in this field of research are common.

Problems of the prior art include that the infection of human cells with the SARS-CoV virus could not effectively be stopped with the currently available pharmaceutical compositions. Problems, furthermore, include that available pharmaceutical compositions and their methods of use cannot safely deliver high doses of antiviral active agents to sites which carry high viral loads during an infection.

Hence, there is currently a significant unfulfilled need for antiviral therapies to prevent and/or treat virus infections, including SARS virus infections such as COVID-19. The previous unsuccessful attempts to develop antiviral treatments against SARS underscore the difficulty to solve this problem. The high number of patients suffering from COVID-19 and the high mortality of the disease underscore the need for an effective treatment.

### SUMMARY OF THE INVENTION

The present invention relates to pharmaceutical compositions and methods for their use in antiviral therapy.

Certain embodiments of the present invention include pharmaceutical compositions comprising at least one active agent from the area of antiviral agents in a pharmaceutically acceptable carrier for administration to the respiratory tract.

One embodiment of the pharmaceutical compositions according to the present invention comprises one or more active agents which inhibit the docking of viruses to cells and/or inhibit the virus activation.

Another embodiment of the pharmaceutical compositions according to the present invention comprises one or more active agents which inhibit the docking of coronaviruses to cells and/or the coronavirus activation.

Yet another embodiment of the pharmaceutical compositions comprises one or more active agents which inhibit the docking of SARS-CoV-2 to cells and/or inhibits the activation of SARS-CoV-2.

Application to the respiratory tract, including but not limited to the administration to the lungs, nose and/or nasal cavities, of the pharmaceutical compositions according to the present invention provides the advantage of safe and targeted delivery of active agents to sites of high viral load.

### DETAILED DESCRIPTION OF THE INVENTION

The present invention will now be described.

The present invention is directed to pharmaceutical compositions and methods for their use in antiviral therapy.

Furthermore, the present invention describes the delivery of pharmaceutical compositions comprising one or more active agents to the respiratory system, including the lungs, the airways, the nose, and the nasal cavities, for example by nasal and/or pulmonal delivery.

Antiviral therapies focus on various specific processes in viral replication, including among others virus entry into host cells (thereby including the processes: virus docking to host cells and virus activation), virus replication in host cells (thereby including the processes: virus RNA translation, proteolysis, translation and RNA replication, and packaging), and virus release from host cells. According to the present invention, active agents targeting different processes of viral replication can be used, including but not limited to inhibitors of virus RNA translation, inhibitors of proteolysis, inhibitors of RNA replication, inhibitors of the docking of viruses to host cell receptors, and inhibitors of virus activation by host cell proteases.

As used herein, the term “active agent” means a compound that, when administered to a patient, confers, directly or indirectly, a physiological effect on the patient. For the present invention, a physiological effect would involve preventing and/or treating conditions like virus infections. Examples of active agents in the context of the present invention are apilimod, aprotinin, arbidol, camostate, hepta arginine (D and L form), hesperidin, iloprost, nafamostat, and remdesivir.

Further examples of active agents according to the present invention include, but are not limited to 4-(4-guanidinobenzoyloxy) phenylacetate, abacavir, acyclovir, adefovir, albuvirtide, amantadine, amprenavir, apricitabine, atazanavir, atovaquone, AT-527, ATR-002, azithromycin, balavir, baloxavir, baricitinib, bemcentinib, bicalutamide, bictegravir, BMS-955176, bequinar, brilacidin, bromhexine, cabotegravir, cenicriviroc, censavudine, cidofovir, clevudine, cobicistat, CS-8958, daclastavir, dalcetrapib, darunavir, decitabine, delaviridine, defibrotide, didanosine, docosanol, dolutegravir, doravirine, edoxudine, EDP1815, efavirenz, elvitegravir, emtricitabine, enfuvirtide, entecavir, etavirine, famiclovir, fosamprenavir, foscarnet, fosfonet, fostemsavir, FT 516, GS-9883, favipiravir, ibacitabine, ibalizumab, ibrutinib, idoxuridine, indinavir, ivermectin, lamivudine, letermovir, leflunomide, lopinavir, loviride, maraviroc, methisazone, molnupiravir, moroxidine, N-(2-aminoethly)-1-aziridine ethane amine, N4-hydroxycytidine (EIDD-1931), nitazoxanide, molnupiravir, oseltamivir, penciclovir, peramivir, piconivir, PCT299, PRO 140, raltegravir, ribavirin, rilprivirine, rimantidine, ritonavir, ruxolitinib, saquinavir, silmatasertib, simeprevir, sofosbuvir, stavudine, telaprevir, telbivudine, tenovir, tenovir alafenamide, tenovir alafenamide fumarate, tenovir disoproxil, teriflunomide, tofacitinib, tipranavir, trifluridine, triazavirin, tromantadine, TXA127, tranexamic acid, ulinastatin, valaciclovir, valganciclovir, VERU-111, viciviroc, vidarabine, viramidine, zalcatibine, zanamivir, and zidovudine.

As used herein the term “virus docking inhibitor” means an active agent that inhibits the docking of viruses to receptors on host cells, like the docking of SARS-CoV-2 with its spikes to converting enzyme 2 (ACE2) receptors on host cells.

As used herein the term “virus activation inhibitor” means an active agent that inhibits the activation of virus protein, like the activation of SARS-CoV-2 spike protein by host cell proteases.

The domain of virus docking inhibitors comprises the class of spike ligands and the class of ACE2 receptor ligands.

As used herein, the term “spike ligand” means an active agent with high affinity to the spike of a virus. Due to the interaction of the spike ligand with the spike of the virus, the docking of the virus to host cells is inhibited. The class of spike ligands comprises various families, including proteins like the recombinant human ACE2 protein and its derivatives; antibodies against spike protein like monoclonal antibodies against the SARS-CoV-2 spike; flavonoids like hesperidin its analogs and derivatives, diosmin its analogs and derivatives, naringin its analogs and derivatives, epigallocatechin gallate its analogs and derivatives, rutin its analogs and derivatives, chrysin its analogs and derivatives; anthraquinones like emodin its analogs and derivatives; prostanoids like iloprost and its derivatives; and substituted indoles like arbidol.

As used herein, the term “ACE2 receptor ligand” means an active agent with high affinity to the ACE2 receptor. Due to the interaction of the ACE2 receptor ligand with the ACE2 receptor of the host cell, the docking of the virus to the host cell is inhibited. The class of ACE2 receptor ligands comprises various families, including antibodies such as monoclonal antibodies against the ACE2 receptor and certain polypeptides including but not limited to angiotensin II its analogs and derivatives.

Due to the interaction of virus activation inhibitors with host cell proteases, including but not limited to furin and serin proteases, virus activation and consequently virus entry into host cells is inhibited.

The domain of virus activation inhibitors comprises the class of furin inhibitors and the class of serin protease inhibitors.

The class of furin inhibitors comprises various families, including but not limited to polypeptides like hexa arginine (D and L form), hepta arginine (D and L form), octa arginine (D and L form), nona argine (D and L form); polyarginine derivatives (D and L form) like hexa-D-arginine amide; and RVKR derivatives like decanoyl—RVKR-chloromethylketone.

The class of serine protease inhibitors comprises various families, including but not limited to bromhexine, ambroxol, and 4-(diaminomethylideneamino)benzoates like camostate and nafamostate, their analogs and derivatives.

Pharmaceutically acceptable salts of the active agents according to the invention can be obtained combining them with suitable anions and cations, depending on the acidic or basic nature of the active agent following procedures known to one skilled in the art and described for example by Wiedmann and Naqwi (S. Wiedmann and A. Naqwi, Pharmaceutical salts: theory, use in solid dosage forms and in situ preparation in an aerosol, Asian J Pharm Sci 11 (2016) 722-734). The aforementioned cations include but are not limited to aluminum, arginine, benzathine, calcium, choloroprocaine, choline, diethanolamine, ethanolamine, ethylene diamine, histidine, lithium, lysine, magnesium, meglumine, potassium, procaine, sodium, trimethylamine, and zinc. The aforementioned anions include but are not limited to acetate, aspartate, benzenesulfonate, benzoate, besylate, bicarbonate, bitartrate, bromide, camsylate, carbonate, chloride, citrate, decanoate, edetate, estolate, esylate, fumarate, gluceptate, gluconate, glutamate, glycolate, glycollylarsanilate, hexanoate, hexylresorcinate, hydrabamine, hydroxynaphtoate, iodide, isothionate, lactate, lactobionate, malate, maleate, mandelate, mesylate, methylbromide, methylnitrate, methylsulfate, mucate, napsylate, nitrate, octanoate, oleate, pamoate, pantothenate, phosphate, polygalacturonate, propionate, salicylate, stearate, subacetate, succinate, sulfate, tartrate, teoclate, tosylate, and triethiodide.

Pharmaceutically acceptable excipients for the pharmaceutical carriers and pharmaceutical compositions described in the present invention include but are not limited to acetic acid, alcohol, anhydrous citric acid, anhydrous trisodium citrate, apaflurane, ascorbic acid, benzalkonium chloride, benzyl alcohol, butylated hydroxyanisole, butylated hydroxytoluene, caffeine, camphor (synthetic), calcium carbonate, calcium chloride, carrageenan, castor oil, cellulose microcrystalline/carboxymethylcellulose, cetylpyridinium chloride, chlorobutanol, citric acid monohydrate, cottonseed oil, dextrose, dextrose monohydrate, dicholorodifluoromethane, dicholortetrafluoroethane, edeteate disodium, eucalyptol, fluorochlorohydrocarbons, gelatin, glycerin, hydrocholoric acid, hydroxyethl cellulose, hypromellose 2906, hypromellose 2910 (15000 MPAS), hypromellose 2910 (4000 MPAS), hypromellose 2910 (5 MPAS), lactose, lactose monohydrate, lanolin, lecithin (soybean), magnesium stearate, mannitol, menthol, methylcellulose, methylparaben, monobasic potassium phosphate, nitric acid, N,N-diemthylacetamide, norflurane, n-phenyl-1-naphtylamine, oleic acid, petrolatum, pectin, phenylethyl alcohol, phenylmercuric acetate, polyethylenglycol 3350, polyethylenglycol 400, polysorbate 20, polysorbate 80, potassium chloride, propylene glycol, propylparaben, saccharin, saccharin sodium, saccharin sodium anhydrous, silicon dioxide, sodium bicarbonate, sodium bisulfite, sodium chloride, sodium hydroxide, sodium lauryl sulfate, sodium phosphate (dibasic) sodium phosphate (dibasic, anhydrous), sodium phosphate (dibasic, dehydrate), sodium phosphate (dibasic, dodecahydrate), sodium phosphate (dibasic, heptahydrate), sodium metabisulfite, sodium sulfate anhydrous, sorbitan monolaurate, sorbitan trioleate, sorbitol, sucralose, sulfuric acid, thiomersal, tricholoromonofluoromethane, trisodium citrate dihydrate, trolamine, tromethamine, zinc oxide, and water.

Currently, there are no FDA-approved antiviral therapies for nasal or pulmonary delivery available. Approved antiviral therapies generally comprise the delivery of pharmaceutical compositions containing active agents orally or intravenously. This holds true even in the case of established therapies for the treatment of respiratory viruses. As such, the current medical practice in treating viral infections is teaching away from alternate routes of administration.

To address the currently unfulfilled need for effective antiviral therapies, including therapies for COVID-19, and to overcome the problems of the prior art, the current invention departs from the well-established routes of application of antiviral treatments, including oral application and intravenous application, and contrarily describes the application of certain pharmaceutical compositions to the respiratory tract, for example by nasal and/or pulmonary delivery, thereby favorably targeting areas of high viral load in the host.

The pharmaceutical compositions according to the present invention include but are not limited to solutions, emulsions, suspensions, and powders. They are applied to the respiratory tract for example directly or via aerosolization using various devices. The application of the pharmaceutical compositions to the respiratory tract follows various regimens, including but not limited to once daily dosing, twice daily dosing, three times per day dosing, four times per day dosing, dosing every other day, once weekly dosing, and continuous dosing.

Pulmonal application according to the present invention is achieved using devices including among others pressurized metered dose inhalers (pMDIs), dry power inhalers (DPIs), soft mist inhalers (SMIs), and nebulizers. Furthermore, these compositions can be administered together with lung surfactants including Curosurf (poractant), Infasurf (calfactant), Survanta (beractant), and Surfactin (lucinactant) among others. In addition, these compositions can be administered to the lung via ventilators including the Evita V600, Evita V800, Evita XL, and Savina 300 (all of Drägerwerk AG & Co. KGaA. Luebeck, Germany). The latter administration can be performed as a co-administration with oxygen.

Several DPIs which are known to one skilled in the art can be utilized to administer the pharmaceutical compositions described by this invention, including among others the Aerosolizer, HandiHaler, Rotohaler, and Neohaler. Various pMDIs which are known to one skilled in the art can also be utilized to administer the pharmaceutical compositions described by this invention, including among others 3M Metered Dose Inhalers and the 3M Intelligent Control Inhaler (3M, St. Paul, Minn.). Furthermore, various SMIs which are known to one skilled in the art can be utilized to administer the pharmaceutical compositions described by this invention, including among others the Respimat Soft Mist Inhaler. In addition, various nebulizers which are known to one skilled in the art can be utilized, including among others the Mabis NebPak Ultrasonic Nebulizer, the Pari Boy (pari.com), the I-neb Adaptive Aerosol Delivery system (https://www.usa.philips.com/healthcare/product/HC85167/i-neb-battery-powered-drug-delivery-system), and the Omron NE-C801 compare Compressor Nebulizer System (https://omronhealthcare.com/products/compair-nebulizer-system-nec801/).

Various nasal applicators of the pressurized type and the non-pressurized type, which are known to one skilled in the art, can be utilized to administer the pharmaceutical compositions described by this invention, including among others the Advanced Preservative Free Nasal Pump (Aptar Pharma; pharma.aptar.com), the Latitude (side-actuated nasal spray device (Aptar Pharma at pharma.aptar.com), E-Lockout (Aptar Pharma; pharma.aptar.com), and the 3M Nasal MDI (3M, St. Paul, Minn.).

Various nasal spray bottles, which are known to one skilled in the art can be utilized to administer the pharmaceutical compositions described by this invention, including among others the ION Sinus (https://ionbiome.com/products/ion-sinus), and XJT nasal spray bottles (Xinjitai Pte. Ltd., 234 Jalan Eunos, Euhabitat, Singapore 415865) of the metered pump type.

Various nasal irrigators, which are known to one skilled in the art can be utilized to administer the pharmaceutical compositions described by this invention, including among others the Navage Nasal Care, the Blue Neti Pot Rinsing Nose Wash System, the Sanvic Professional Nasal/Sinus Pulsatile Irrigator (https://www.medexsupply.com/personal-care-general-living-aids-sanvic-pulsatile-nasal-irrigator-with-2-adult-1-child-nasal-tip-x_pid-82772.html), and the NeilMed Singator.

Pharmaceutical compositions according to the present invention can be combined with one or more additional treatments, including pharmaceutical compositions and/or active agents for oral and/or intravenous delivery, to prevent and/or treat virus infections. Combination administration according to the present invention can be carried out simultaneously and/or sequentially.

One embodiment of a combination treatment according to the present invention is the administration of a pharmaceutical composition containing 100 mg camostate mesylate to the respiratory tract of a patient as described in Example 2 below, combined with the intravenous administration of 100 mg remdesivir over 30 to 120 minutes once a day. As known to one skilled in the art, the doses of the combination administration can be adjusted to the needs of the individual patient and can be modified over the course of a multi-day combination administration.

The present invention will hereinafter be described with reference to exemplary embodiments, which are written to be understood only as examples and are not intended to limit the scope of the present application. The manufacturing processes described below can be scaled up to commercial scales using manufacturing equipment and technologies known to one of ordinary skill in the art.

### EXAMPLES

### Example 1

250 mg of hesperidin (Sigma Aldrich; micronized by jet milling using a Jet-O-Mizer Model 00 (Fluid Energy Processing and Equipment Company, Telford, Pa., USA) to obtain a median diameter of 4 μm) are suspended in 5 mL of a 2% polysorbate 80 (Tween 80 HP, Croda) solution in water.

(Alternatively, 5 ml 0.9% sodium chloride solution in water (ADDIPAK Saline Solution Unit Dose, 0.9% sodium chloride, 5 mL) can be used to suspend the 250 mg of hesperidin.)

The resulting suspension is placed in an OMRON NE-C801 Compressor Nebulizer system.

The hesperidin suspension is inhaled over a period of up to 20 minutes.

### Example 2

100 mg camostate mesylate (Sigma Aldrich; micronized by jet milling using a Jet-O-Mizer Model 00 (Fluid Energy Processing and Equipment Company, Telford, Pa., USA) to obtain a median diameter of 2 μm) are suspended in 5 mL of a 2% polysorbate 80 (Tween 80 HP, Croda) solution in water.

(Alternatively, 5 ml 0.9% sodium chloride solution in water (ADDIPAK Saline Solution Unit Dose, 0.9% sodium chloride, 5 mL) can be used to suspend the 100 mg of camostate mesylate.)

The resulting suspension is placed in an OMRON NE-C801 Compressor Nebulizer system.

The camostate mesylate suspension is inhaled over a period of up to 20 minutes.

### Example 3

100 mg camostate mesylate (Sigma Aldrich; micronized by jet milling using a Jet-O-Mizer Model 00 (Fluid Energy Processing and Equipment Company, Telford, Pa., USA)) to obtain a median diameter of 2 μm) and 100 mg arbidol hydrochloride (Sigma Aldrich; micronized by jet milling using a Jet-O-Mizer Model 00 (Fluid Energy Processing and Equipment Company, Telford, Pa., USA) to obtain a median diameter of 2 μm) are suspended in 5 mL of a 2% polysorbate 80 (Tween 80 HP, Croda) solution in water. (Alternatively, 5 ml 0.9% sodium chloride solution in water (ADDIPAK Saline Solution Unit Dose, 0.9% sodium chloride, 5 mL) can be used to suspend the 100 mg of camostate mesylate.)

The resulting suspension is placed in an OMRON NE-C801 Compressor Nebulizer system.

The suspension containing camostate mesylate and arbidol hydrochloride is inhaled over a period of up to 20 minutes. The application is performed twice per day.

### Example 4

50 mg remdesivir is dissolved in 10 mL 0.9% sodium chloride solution in water and filled into an ION Sinus spray bottle for subsequent nasal application. The resulting suspension is applied by 10 actuations (five actuations per nostril, 0.1 mL per actuation). The application is performed three times per day.

### Example 5

250 mg arbidol (Sigma Aldrich; micronized by jet milling using a Jet-O-Mizer Model 00 (Fluid Energy Processing and Equipment Company, Telford, Pa., USA)) to obtain a median diameter of 15 μm) are suspended in 250 mL 0.9% sodium chloride solution in water. The resulting suspension is placed into a Sanvic Professional Nasal/Sinus Pulsatile Irrigator and applied to rinse the nose and nasal cavities over a period of up to 10 minutes.

### Example 6

1 mg of bromhexine hydrochloride is dissolved in 5 mL of water (Alternatively, 5 ml 0.9% sodium chloride solution in water (ADDIPAK Saline Solution Unit Dose, 0.9% sodium chloride, 5 mL) can be used to dissolve the 1 mg of bromhexine hydrochloride.)

The resulting solution is placed in an I-neb Adaptive Aerosol Delivery system.

The bromhexine hydrochloride solution is inhaled over a period of up to 15 minutes.

The invention being thus described, it will be obvious that the same may be varied in many ways. Such variations are not to be regarded as a departure from the spirit and scope of the invention and all such modifications as would be obvious to one skilled in the art are intended to be included within the scope of the following claims.

1. A pharmaceutical composition comprising at least one active agent from the area of antiviral agents in a pharmaceutically acceptable carrier for administration to the respiratory tract.

2. The pharmaceutical composition according to claim 1, comprising at least one active agent from the domains of virus docking inhibitors and/or virus activation inhibitors.

3. The pharmaceutical composition according to claim 1, comprising a flavonoid alone or in combination with one or more antiviral agents in a pharmaceutically acceptable carrier for administration to the respiratory tract.

4. The pharmaceutical composition according to claim 1, comprising hesperidin alone or in combination with one or more antiviral agents in a pharmaceutically acceptable carrier for administration to the respiratory tract.

5. The pharmaceutical composition according to claim 1, for the prevention and/or treatment of infections with corona viruses.

6. The pharmaceutical composition according to claim 1, for the prevention and/or treatment of COVID-19.

7. A method of use whereby the pharmaceutical composition according to claim 1 is applied to the lungs.

8. A method of use whereby the pharmaceutical composition according to claim 1 is applied to the nose and/or nasal cavities.
